(19) United States
(12) Patent Application Publication
(10) Pub. No.: US 2024/0237932 A1
(43) Pub. Date: Jul. 18, 2024

(54) Biological Information Measurement System

(71) Applicant: Shimadzu Corporation, Kyoto-shi (JP)

(72) Inventors: Masafumi FURUTA, Kyoto-shi (JP); Yoshihiro INOUE, Kyoto-shi (JP); Shin NAKAMURA, Kyoto-shi (JP)

(21) Appl. No.: 18/028,949

(22) PCT Filed: Sep. 9, 2021
(86) PCT No.: PCT/JP2021/033118

§ 371 Date: Mar. 28, 2023

(30) Foreign Application Priority Data
Sep. 29, 2020 (JP) 2020-162869

(51) Int. Cl. A61B 5/18; A61B 5/00; A61B 5/01; A61B 5/0205; A61B 5/024; A61B 5/026; A61B 5/08; A61B 5/11; A61B 5/16; A61B 5/243; A61B 5/256; A61B 5/291

(52) U.S. Cl. CPC A61B 5/18; A61B 5/0077; A61B 5/02055; A61B 5/0261; A61B 5/165; A61B 5/243; A61B 5/256; A61B 5/291; A61B 5/4064; A61B 5/6803; A61B 5/6831; A61B 5/7405; A61B 5/742; A61B 5/01; A61B 5/024; A61B 5/08; A61B 5/11; A61B 2503/20; A61B 2560/045; A61B 2562/0204; A61B 2562/0219; A61B 2562/0271

(57) ABSTRACT

A physiological information measurement system capable of achieving decrease in time for attachment of a plurality of sensors to a subject and preparation for measurement is provided. A head-mounted measurement system includes a housing attachable to and removable from a head of a subject, a plurality of sensors held by the housing, and a control unit (signal processing unit) that synchronizes signals outputted from the plurality of sensors with each other. The plurality of sensors include an NIRS probe (a first sensor) that measures brain activities and an ultra-wide-angle camera (a second sensor) that obtains an image of a face of the subject.

### TECHNICAL FIELD

The present disclosure relates to a physiological information measurement system that measures brain activities.

### BACKGROUND ART

In order to provide goods and services in consideration of feelings of individuals, a system that can analyze information on feelings of a subject has recently been proposed. For example, PTL 1 discloses an apparatus that automatically obtains evaluation of a sample (wine) by shooting moving images of a subject who tries the sample with the use of a camera provided in a wine dispenser where the sample is contained and analyzing the subject's facial expression from the moving images to estimate the subject's feeling.

### CITATION LIST

### Patent Literature

PTL 1: Japanese Patent Laying-Open No. 2018-106419

### SUMMARY OF INVENTION

### Technical Problem

In order to estimate information on sensibility of a subject, a plurality of types of sensors are preferably used. For example, as described in Japanese Patent Laying-Open No. 2019-159941, in addition to a camera that shoots a subject's face, another sensor that obtains physiological information of the subject may be used. In this literature, a face of a worker on production lines of a factory is shot with a camera, an electroencephalograph is attached to a head of the worker, and a degree of concentration of the worker is estimated from a shot image and outputs from the electroencephalograph.

In general, ease in attachment to a subject, of a sensor that measures brain activities is an issue. There is a room for improvement in a method of efficiently obtaining physiological information of a subject with a plurality of types of sensors including such a sensor as measuring brain activities.

The present disclosure was made to solve such a problem, and provides a physiological information measurement system that achieves improved ease in attachment to a subject, of a plurality of types of sensors including a sensor that measures brain activities.

### Solution to Problem

A physiological information measurement system in the present disclosure includes a base attachable to and removable from a head of a subject, a plurality of sensors held by the base, and a signal processing unit that synchronizes signals outputted from the plurality of sensors. The plurality of sensors include a first sensor that measures brain activities and a second sensor that obtains an image of a face of the subject.

### Advantageous Effects of Invention

According to the physiological information measurement system, a plurality of sensors are held by the base removably fixed to the head of the subject, so that ease in attachment to the subject is improved. Furthermore, since the second sensor can obtain an image of the facial expression of the subject, the image of the facial expression of the subject can be obtained without being affected by a motion of the subject.

### BRIEF DESCRIPTION OF DRAWINGS

FIG. 1 is a diagram showing an exemplary entire construction of a head-mounted measurement system according to a first embodiment.

FIG. 2 is a block diagram for illustrating a configuration of a head-mounted device of the head-mounted measurement system according to the first embodiment.

FIG. 3 is a diagram for illustrating a range of shooting by an image apparatus provided in the head-mounted measurement system according to the first embodiment.

FIG. 4 is a diagram showing an exemplary entire construction of a head-mounted measurement system according to a second embodiment.

FIG. 5 is a block diagram for illustrating a configuration of a signal processing apparatus of the head-mounted measurement system according to the second embodiment.

### DESCRIPTION OF EMBODIMENTS

The present embodiment will be described in detail below with reference to the drawings. The same or corresponding elements in the drawings have the same reference characters allotted and description thereof will not be repeated.

### First Embodiment

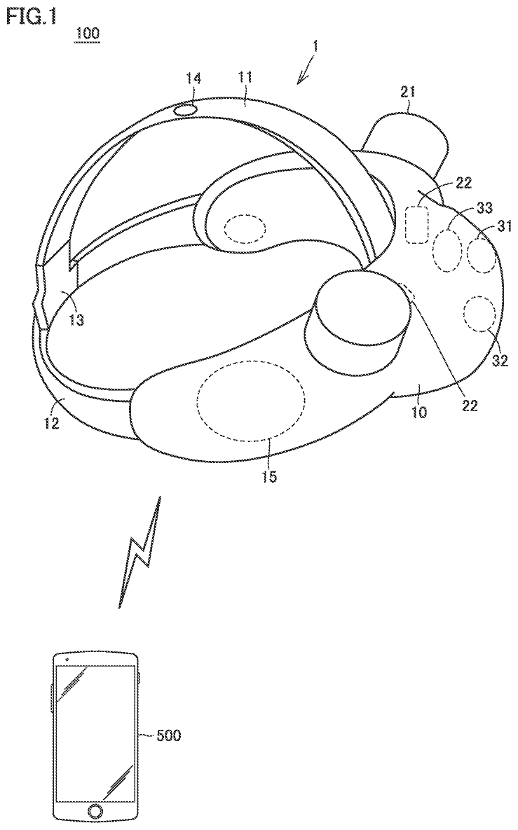

A head-mounted measurement system that measures brain activities will be described below as an exemplary physiological information measurement system. FIG. 1 is a diagram showing an exemplary entire construction of a head-mounted measurement system 100 according to a first embodiment. Head-mounted measurement system 100 is a system including at least a head-mounted device 1 to which a sensor that measures brain activities is attached. Head-mounted device 1 includes a housing 10 in which a plurality of sensors are held and a fixing portion that removably fixes housing 10 to a head of a subject. Housing 10 should only be a base attachable to and removable from the head of the subject, and it is not limited to a housing in a box shape. Though a construction in which the fixing portion is provided in housing 10 will be described below, a construction in which a fixing portion such as a band is not provided but housing 10 is attachable to and removable from the head of the subject by making use of a head curvature of an upper portion of the forehead may be applicable.

The fixing portion shown in FIG. 1 includes a vertex band 11 for prevention of slip-off, a head band 12 that fixes a position of housing 10, and a size adjustment portion 13 that allows adjustment of a length of vertex band 11 and head band 12 in accordance with a size of the head. Size adjustment portion 13 should only allow adjustment of the length of at least head band 12. Head-mounted device 1 and the fixing portion shown in FIG. 1 are by way of example. Head-mounted device 1 may have such a geometry as allowing coverage from the head to a neck portion of the subject, and any structure of the fixing portion may be applicable so long as the position of housing 10 can be fixed in conformity with the shape of head-mounted device 1. In the present disclosure, the expression as the head below may encompass not only the head but also the neck portion.

A sensor that measures brain activities is attached to head-mounted device 1. In order to correctly measure brain activities, the sensor should be arranged at a prescribed position of the head of the subject. Therefore, for arrangement of the sensor at the prescribed position of the head of the subject at the time of attachment of head-mounted device 1 to the head of the subject, an alignment portion 14 is provided in vertex band 11. By bringing alignment portion 14 in alignment with a vertex point of the subject, the sensor is arranged at the prescribed position of the head of the subject. By providing alignment portion 14, anybody including the subject himself/herself can easily attach head-mounted device 1 to a correct position.

A sensor (first sensor) that measures brain activities and an ultra-wide-angle camera 31 (second sensor) which is an image sensor that obtains an image of the face of the subject are attached to housing 10. Ultra-wide-angle camera 31 is arranged at a position out of a field of view of the subject. For example, housing 10 is provided with a protruding portion that protrudes forward from a frontal portion of the subject, and as ultra-wide-angle camera 31 is attached to the protruding portion, it can be arranged at a position outside the field of view of the subject. By thus arranging ultra-wide-angle camera 31 out of the field of view of the subject, ultra-wide-angle camera 31 does not block a line of sight of the subject. Examples of the sensor that measures brain activities include an NIRS probe 21 that measures a cerebral blood flow with near-infrared spectroscopy (NIRS), a brain wave sensor 22 (EEG), and a magnetoencephalograph (MEG). Though NIRS probe 21 and brain wave sensor 22 are attached to housing 10 shown in FIG. 1, another sensor that measures brain activities may further be provided. Housing 10 may be constructed such that any one of NIRS probe 21 and brain wave sensor 22 is attached.

NIRS probe 21 is a sensor that non-invasively measures variation in amount of a cerebral blood flow with the use of near infrared light having such a property as passing through a skin tissue or a bone tissue but being absorbed by oxyhemoglobin and deoxyhemoglobin in blood. NIRS probe 21 detects signal light by emitting near infrared light from a plurality of light transmission portions (light transmission fibers) arranged at prescribed intervals toward a scalp of the subject and receiving light diffused and reflected by the brain (cerebral cortex) with a plurality of light reception portions (light reception fibers) on the scalp. A concentration of oxyhemoglobin and a concentration of deoxyhemoglobin at a measurement site located between the light transmission portions and the light reception portions are found based on this detection light. Brain activities are then measured based on variation in concentration of hemoglobin.

In head-mounted device 1 shown in FIG. 1, NIRS probe 21 is provided on each of the left and the right of the frontal portion of the subject to measure cerebral blood flows in a dorsolateral prefrontal area of the subject. Single NIRS probe 21 is provided with a measurement unit with four channels, and head-mounted device 1 can measure cerebral blood flows with the use of measurement units with eight channels in total. NIRS probe 21 is provided with a hair thrusting function to thrust hairs aside with a rod-shaped tool to expose the surface of the head at a position of attachment of the sensor so as to prevent measurement light from being blocked by hairs in a case where the NIRS probe is arranged in a portion with hairs in the head of the subject (a site with hairs).

Head-mounted device 1 shown in FIG. 1 measures cerebral blood flows in the dorsolateral prefrontal area of the subject to allow studies about brain activities such as cognition by the subject. When other brain activities of the subject are to be studied, for example, when brain activities in connection with the subject's feelings are to be studied, however, NIRS probe 21 is newly inserted in a receptacle 15 located at another position of housing 10. By newly inserting NIRS probe 21 into receptacle 15, NIRS probe 21 can measure cerebral blood flows in a temporal portion of the subject. Housing 10 thus includes a mechanism that allows change in position where NIRS probe 21 is to be held by housing 10 in accordance with a brain activity to be measured (for example, cognition, sensibility, or the like). The mechanism for change in position is not limited to receptacle 15.

Brain wave sensor 22 is a sensor that obtains a weak current generated by activities in the brain with an electrode attached to a skull, amplifies the current, and measures the current as brain waves. A shape or a configuration of brain wave sensor 22 is not particularly limited so long as the brain wave sensor is capable of measuring brain waves. Housing 10 may include a mechanism that allows change in position where brain wave sensor 22 is to be held by housing 10 in accordance with a brain activity to be measured.

Not only ultra-wide-angle camera 31 but also other sensors such as a temperature sensor 32 capable of measuring a body temperature of the subject and an acceleration sensor 33 that measures a motion of the head of the subject may be attached to housing 10 as the second sensor. Other sensors may include any sensor so long as the sensor measures a target different from the target of the sensor that measures brain activities, and a sensor that detects a motion of the subject, a sensor that measures a heartbeat, and a sensor that measures respiration may be applicable. Though temperature sensor 32 and acceleration sensor 33 other than ultra-wide-angle camera 31 are attached to housing 10 in head-mounted device 1 shown in FIG. 1, other sensors may further be attached to housing 10. Furthermore, head-mounted device 1 may be configured such that ultra-wide-angle camera 31 is not attached to housing 10 but temperature sensor 32 and acceleration sensor 33 other than the sensor that measures brain activities may be attached to housing 10.

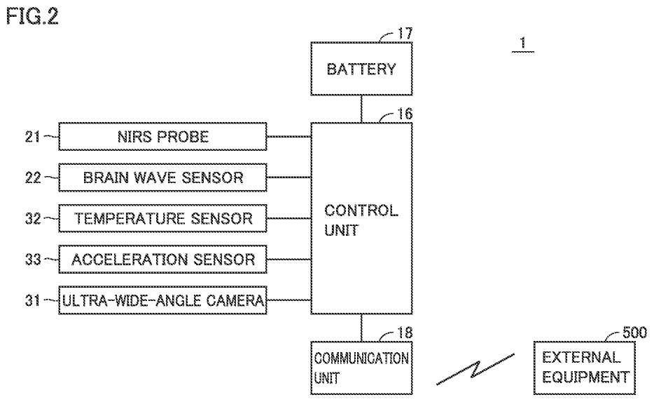

Other than NIRS probe 21, brain wave sensor 22, ultra-wide-angle camera 31, temperature sensor 32, and acceleration sensor 33, housing 10 is provided with a control unit that controls these sensors. FIG. 2 is a block diagram for illustrating a configuration of the head-mounted device of the head-mounted measurement system according to the first embodiment. As shown in FIG. 2, NIRS probe 21, brain wave sensor 22, ultra-wide-angle camera 31, temperature sensor 32, and acceleration sensor 33 are connected to a control unit 16. A battery 17 as a power supply for drive and a communication unit 18 for communication with external equipment 500 such as a smartphone are further connected to control unit 16. Battery 17 and communication unit 18 are provided in housing 10. In head-mounted device 1, battery 17 and communication unit 18 may naturally be provided outside housing 10.

Control unit 16 includes a processor such as a central processing unit (CPU) and a memory such as a read only memory (ROM) and a random access memory (RAM). A control program is stored in the memory. As the processor executes the control program, it controls such sensors as NIRS probe 21 and ultra-wide-angle camera 31.

Control unit 16 performs signal processing for synchronizing a signal (first signal) resulting from measurement by NIRS probe 21 with a signal (second signal) resulting from image obtainment by ultra-wide-angle camera 31. In other words, control unit 16 corresponds to a signal processing unit that performs signal processing for synchronizing the signal (first signal) resulting from measurement by NIRS probe 21 with the signal (second signal) resulting from image obtainment by ultra-wide-angle camera 31, and it is provided in housing 10. Control unit 16 synchronizes the signal resulting from measurement by NIRS probe 21 with the signal resulting from image obtainment by ultra-wide-angle camera 31 based on time stamps of these signals. Signal processing for synchronizing the signals is not limited to processing based on the time stamp of the signal. Control unit 16 may generate a synchronization signal and supply the synchronization signal to sensors to synchronize signals from the sensors with each other.

Battery 17 may be, for example, a secondary battery such as a nickel metal hydride battery or a lithium ion secondary battery or a primary battery such as a dry cell. Naturally, head-mounted device 1 may be provided with no battery 17, or may be driven by a commercial power supply together with battery 17.

Communication unit 18 is a communication interface, and can communicate with external equipment 500 by establishing wireless or wired communication. Therefore, external equipment 500 can receive through communication unit 18, the signal (first signal) resulting from measurement by NIRS probe 21 and the signal (second signal) resulting from image obtainment by ultra-wide-angle camera 31 as signals synchronized by control unit 16. Since the signal resulting from image obtainment by ultra-wide-angle camera 31 includes a signal resulting at least from image obtainment of the facial expression of the subject, external equipment 500 can obtain the facial expression of the subject and the signal resulting from measurement of brain activities at that time (the signal resulting from measurement by NIRS probe 21 and the signal resulting from measurement by brain wave sensor 22). Therefore, a researcher can study relation between delight, anger, sorrow and pleasure emotions of the subject and the brain activities based on the signals obtained by external equipment 500. In addition, the researcher can know a state (for example, sleeping, being nervous, or the like) of the subject at the time point of measurement of the brain activities based on the signals obtained by external equipment 500 and then study the brain activities.

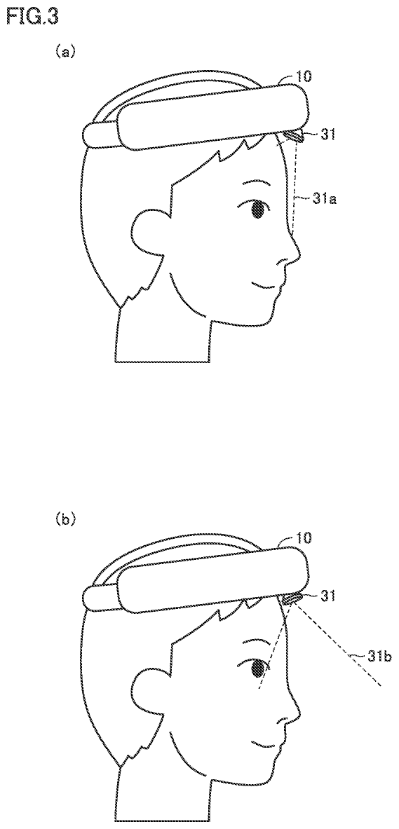

The signal resulting from image obtainment by ultra-wide-angle camera 31 is described as including the signal resulting at least from image obtainment of the facial expression of the subject. In other words, a range of shooting by ultra-wide-angle camera 31 includes the subject's face without fail. FIG. 3 is a diagram for illustrating a range of shooting by an image apparatus provided in the head-mounted measurement system according to the first embodiment. Since ultra-wide-angle camera 31 is attached as facing the subject in FIG. 3 (a), a range of shooting 31a of ultra-wide-angle camera 31 includes only the subject's face. Therefore, in the case of ultra-wide-angle camera 31 shown in FIG. 3 (a), the signal resulting from image obtainment by ultra-wide-angle camera 31 consists of a signal resulting from image obtainment of the facial expression of the subject.

Since ultra-wide-angle camera 31 is attached as facing the outside of the subject in FIG. 3 (b), a range of shooting 31b of ultra-wide-angle camera 31 includes not only the subject's face but also an operation performed by the subject and an environment around the subject. Therefore, in the case of ultra-wide-angle camera 31 shown in FIG. 3 (b), the signal resulting from image obtainment by ultra-wide-angle camera 31 includes also a signal resulting from image obtainment of an operation performed by the subject and a signal resulting image obtainment of an environment around the subject other than the signal resulting from image obtainment of the facial expression of the subject. The environment around the subject includes an object or an event that the subject sees, a person with whom the subject talks, or an animal with which the subject interacts.

As a result of synchronization between the signal resulting from image obtainment of the operation performed by the subject and the signal resulting from measurement of brain activities (the signal resulting from measurement by NIRS probe 21 and the signal resulting from measurement by brain wave sensor 22), external equipment 500 can obtain the operation performed by the subject and the signal resulting from measurement of brain activities at that time. Therefore, the researcher can study about relation between an action of the subject and the brain activities based on the signals obtained by external equipment 500. In addition, the researcher can know a behavior (for example, wiggling a leg or the like) of the subject at the time point of measurement of the brain activities based on the signals obtained by external equipment 500 and then study about the brain activities.

As a result of synchronization between the signal resulting from image obtainment of the environment around the subject and the signal resulting from measurement of brain activities (the signal resulting from measurement by NIRS probe 21 and the signal resulting from measurement by brain wave sensor 22), external equipment 500 can obtain the environment around the subject and the signal resulting from measurement of brain activities at that time. Therefore, the researcher can study about relation between the environment (for example, a facial expression of a person with whom the subject talks) around the subject and the brain activities based on the signals obtained by external equipment 500. In addition, the researcher can know the environment (for example, playing with a pet) around the subject at the time point of measurement of the brain activities based on the signals obtained by external equipment 500 and then study about the brain activities.

Though ultra-wide-angle camera 31 is described as being used as the image sensor, an image sensor to be attached to head-mounted device 1 is not limited to ultra-wide-angle camera 31. Head-mounted device 1 may be provided, for example, with two image sensors of an image sensor that obtains an image of a facial expression of the subject and an image sensor that obtains an image of an environment around the subject. Alternatively, head-mounted device 1 may include a mechanism capable of moving a single image sensor to a position for image obtainment of a facial expression of the subject or to a position for image obtainment of an environment around the subject to have the image sensor obtain an image.

In the description above, control unit 16 is described as performing processing for synchronizing the signal (first signal) resulting from measurement by NIRS probe 21 with the signal (second signal) resulting from image obtainment by ultra-wide-angle camera 31. Control unit 16, however, may not only synchronize the signal (first signal) resulting from measurement by NIRS probe 21 with the signal (second signal) resulting from image obtainment by ultra-wide-angle camera 31 but also may generate a signal associated with cognition or sensibility obtained from the synchronized signals and transmit the signal to external equipment 500 through communication unit 18.

In the description above, control unit 16 is described as performing processing for synchronizing the signal (first signal) resulting from measurement by NIRS probe 21 with the signal (second signal) resulting from image obtainment by ultra-wide-angle camera 31. Control unit 16, however, may perform processing for synchronizing a signal (second signal) resulting from measurement by temperature sensor 32 or acceleration sensor 33 other than the signal resulting from image obtainment by ultra-wide-angle camera 31 with the signal (first signal) resulting from measurement by NIRS probe 21. External equipment 500 can thus obtain the signal resulting from measurement by temperature sensor 32 or acceleration sensor 33 and the signal resulting from measurement of brain activities (the signal resulting from measurement by NIRS probe 21 and the signal resulting from measurement by brain wave sensor 22) at that time, rather than the signal resulting from image obtainment by ultra-wide-angle camera 31.

### Second Embodiment

Head-mounted measurement system 100 according to the first embodiment is described as performing processing for synchronizing the signal (first signal) resulting from measurement by NIRS probe 21 with the signal (second signal) resulting from image obtainment by ultra-wide-angle camera 31 by means of control unit 16 provided in housing 10 of head-mounted device 1. In a head-mounted measurement system according to a second embodiment, processing for synchronizing the signal (first signal) resulting from measurement by the NIRS probe with the signal (second signal) resulting from image obtainment by the ultra-wide-angle camera is performed in an apparatus different from the housing of the head-mounted device.

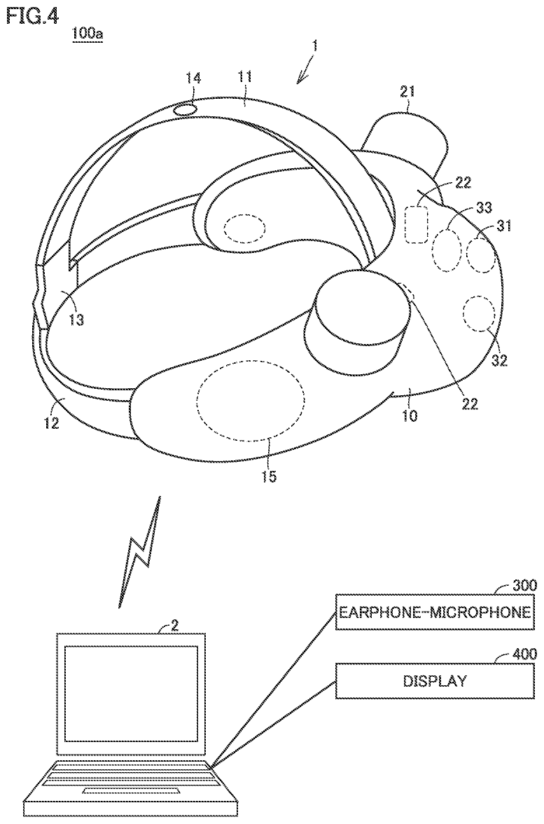

FIG. 4 is a diagram showing an exemplary entire construction of a head-mounted measurement system 100a according to the second embodiment. Head-mounted measurement system 100a is a system including at least head-mounted device 1 to which the sensor that measures brain activities is attached and a signal processing apparatus 2 capable of communicating with head-mounted device 1. Head-mounted device 1 includes housing 10 in which a plurality of sensors are held and the fixing portion that removably fixes housing 10 to the head of a subject. Since head-mounted device 1 shown in FIG. 4 is the same in construction as head-mounted device 1 described in the first embodiment, the same components have the same reference characters allotted and detailed description thereof will not be repeated. In head-mounted device 1 in the second embodiment, processing for

synchronizing the signal (first signal) resulting from measurement by NIRS probe 21 with the signal (second signal) resulting from image obtainment by ultra-wide-angle camera 31 is not performed by control unit 16 but the signals are merely transmitted to signal processing apparatus 2 through communication unit 18.

Signal processing apparatus 2 performs processing for synchronizing the signal (first signal) resulting from measurement by NIRS probe 21 with the signal (second signal) resulting from image obtainment by ultra-wide-angle camera 31 that are received from head-mounted device 1. In other words, signal processing apparatus 2 corresponds to the signal processing unit that performs signal processing for synchronizing the signal (first signal) resulting from measurement by NIRS probe 21 with the signal (second signal) resulting from image obtainment by ultra-wide-angle camera 31, and it is provided in an apparatus different from housing 10. Signal processing apparatus 2 synchronizes the signal resulting from measurement by NIRS probe 21 with the signal resulting from image obtainment by ultra-wide-angle camera 31 based on time stamps of these signals. Signal processing for synchronizing the signals is not limited to processing based on the time stamp of the signal.

Signal processing apparatus 2 may not only synchronize the signal (first signal) resulting from measurement by NIRS probe 21 with the signal (second signal) resulting from image obtainment by ultra-wide-angle camera 31 but also may generate a signal associated with cognition or sensibility obtained from the synchronized signals.

Signal processing apparatus 2 may perform processing for synchronizing the signal (second signal) resulting from measurement by temperature sensor 32 or acceleration sensor 33 other than the signal resulting from image obtainment by ultra-wide-angle camera 31 with the signal (first signal) resulting from measurement by NIRS probe 21. Signal processing apparatus 2 can thus obtain the signal resulting from measurement by temperature sensor 32 or acceleration sensor 33 and the signal resulting from measurement of brain activities (the signal resulting from measurement by NIRS probe 21 and the signal resulting from brain wave sensor 22) at that time, rather than the signal resulting from image obtainment by ultra-wide-angle camera 31.

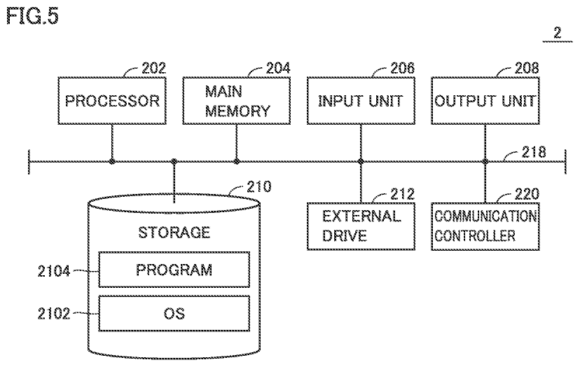

FIG. 5 is a block diagram for illustrating a configuration of signal processing apparatus 2 of head-mounted measurement system 100a according to the second embodiment. Referring to FIG. 5, signal processing apparatus 2 includes a processor 202, a main memory 204, an input unit 206, an output unit 208, a storage 210, an external drive 212, and a communication controller 220. These components are connected to one another through a processor bus 218.

Processor 202 is implemented by a CPU or a GPU, and can read a program (by way of example, an OS 2102 and a program 2104) stored in storage 210, develop the program on main memory 204, and execute the program. Processor 202 executes program 2104 for synchronizing a signal from head-mounted device 1 which is received by input unit 206.

Main memory 204 is implemented by a volatile storage device such as a DRAM or an SRAM. Storage 210 is implemented, for example, by a non-volatile storage device such as an HDD or an SSD.

In addition to OS 2102 for performing a basic function, program 2104 for providing a function as signal processing apparatus 2 is stored in storage 210. In other words, as program 2104 is executed by processor 202 of signal processing apparatus 2, processing for synchronizing signals or generating a signal associated with cognition or sensibility obtained from the synchronized signals is performed.

Input unit 206 includes an input interface connected to head-mounted device 1 wirelessly or through a wire to receive a signal from head-mounted device 1. Input unit 206 can be implemented by a keyboard or a mouse and can further receive an operation from a measurer.

Output unit 208 includes an output interface that outputs to external equipment through communication or USB connection, a signal synchronized by processor 202 or a signal associated with cognition or sensibility which is generated from synchronized signals. Output unit 208 can output a result of processing by processor 202 to a display, various indicators, or a printer connected to signal processing apparatus 2, and can further output a signal to an earphone-microphone 300 for giving an auditory stimulus to the subject or output a signal to display 400 for giving a visual stimulus to the subject.

Signal processing apparatus 2 includes external drive 212, which allows reading of a program stored in a recording medium (for example, an optical recording medium such as a digital versatile disc (DVD) or a USB memory) in which a computer readable program is stored and installation of the program in storage 210 or the like. Communication controller 220 exchanges a signal with another apparatus or the like through wired or wireless communication. Signal processing apparatus 2 may exchange with head-mounted device 1 through communication controller 220, synchronized signals or a signal associated with cognition or sensibility generated from the synchronized signals.

Though program 2104 or the like executed by signal processing apparatus 2 may be installed through a computer readable recording medium, it may be installed by being downloaded from a server apparatus on a network. A function provided by signal processing apparatus 2 may be implemented by using a part of a module provided by the OS.

Signal processing apparatus 2 may present an audio stimulus to the subject with the use of earphone-microphone 300 (an audio device) shown in FIG. 4. As signal processing apparatus 2 presents the audio stimulus to the subject, signal processing apparatus 2 can synchronize timing of presentation of the audio stimulus, the signal (first signal) resulting from measurement by NIRS probe 21, and the signal (second signal) resulting from image obtainment by ultra-wide-angle camera 31 with one another. Signal processing apparatus 2 can obtain the facial expression of the subject at the time of presentation of the audio stimulus to the subject and the signal resulting from measurement of brain activities (the signal resulting from measurement by NIRS probe 21 and the signal resulting from measurement by brain wave sensor 22). Therefore, the researcher can study about relation between a reaction of the subject to the audio stimulus and the brain activities based on the signals obtained by signal processing apparatus 2. Earphone-microphone 300 may be provided separately from or integrally with head-mounted device 1.

Signal processing apparatus 2 may present a visual stimulus to the subject with the use of display 400 (a display device) shown in FIG. 4. As signal processing apparatus 2 presents the visual stimulus to the subject, signal processing apparatus 2 can synchronize timing of presentation of the visual stimulus, the signal (first signal) resulting from measurement by NIRS probe 21, and the signal (second signal) resulting from image obtainment by ultra-wide-angle camera 31 with one another. Signal processing apparatus 2 can obtain the facial expression of the subject at the time of presentation of the visual stimulus to the subject and the signal resulting from measurement of brain activities. Therefore, the researcher can study about relation between a reaction of the subject to the visual stimulus and the brain activities based on the signals obtained by signal processing apparatus 2. Display 400 may be a liquid crystal display provided separately from head-mounted device 1 or a head-mounted display provided integrally with head-mounted device 1.

Earphone-microphone 300 may be used as a voice collector that collects oral communication between the subject and the measurer. Signal processing apparatus 2 can synchronize the oral communication between the subject and the measurer collected by earphone-microphone 300, the signal (first signal) resulting from measurement by NIRS probe 21, and the signal (second signal) resulting from image obtainment by ultra-wide-angle camera 31 with one another. Signal processing apparatus 2 can obtain the facial expression of the subject at the time of conversation with the subject and the signal resulting from measurement of brain activities. Therefore, the researcher can study about relation between contents of the conversation with the subject and the brain activities based on the signals obtained by signal processing apparatus 2.

Signal processing apparatus 2 is described as synchronizing the timing of presentation of the audio stimulus or the visual stimulus, the signal (first signal) resulting from measurement by NIRS probe 21, and the signal (second signal) resulting from image obtainment by ultra-wide-angle camera 31 with one another. Without being limited as such, as described in the first embodiment, control unit 16 provided in housing 10 of head-mounted device 1 may synchronize the timing of presentation of the audio stimulus or the visual stimulus, the signal (first signal) resulting from measurement by NIRS probe 21, and the signal (second signal) resulting from image obtainment by ultra-wide-angle camera 31 with one another.

### Aspects

The embodiments described above are understood by a person skilled in the art as specific examples of aspects below.

### Clause 1

A physiological information measurement system according to one aspect includes a base attachable to and removable from a head of a subject, a plurality of sensors held by the base, and a signal processing unit that synchronizes signals outputted from the plurality of sensors. The plurality of sensors include a first sensor that measures brain activities and a second sensor that obtain an image of a face of the subject.

According to the physiological information measurement system described in Clause 1, as the plurality of sensors are held by the base removably fixed to the head of the subject, ease in attachment to the subject is improved. Furthermore, since the second sensor can obtain the image of the facial expression of the subject, the image of the facial expression of the subject can be obtained without being affected by a motion of the subject.

### Clause 2

In the physiological information measurement system described in Clause 2, the second sensor is arranged at a position out of a field of view of the subject.

According to the physiological information measurement system described in Clause 2, the second sensor does not block the line of sight of the subject.

### Clause 3

In the physiological information measurement system described in Clause 3, the base includes a protruding portion that protrudes forward from a frontal portion of the subject, and the second sensor is attached to the protruding portion.

According to the physiological information measurement system described in Clause 3, the second sensor can be arranged at a position where it does not block the line of sight of the subject.

### Clause 4

The physiological information measurement system described in Clause 4 includes a fixing portion that removably fixes the base to the head of the subject. The fixing portion includes a vertex band for prevention of slip-off, a head band that fixes a position of the base, and a size adjustment portion that allows adjustment of a length of at least the head band in accordance with a size of the head.

According to the physiological information measurement system described in Clause 4, the base can reliably be fixed to the head of the subject without slipping from the head of the subject.

### Clause 5

In the physiological information measurement system described in Clause 5, the vertex band includes an alignment portion aligned with a vertex point.

According to the physiological information measurement system described in Clause 5, the base can easily be attached such that the first sensor and the second sensor are arranged at prescribed positions (correct positions) in the head of the subject.

### Clause 6

In the physiological information measurement system described in Clause 6, the base includes a mechanism that changes a position where the first sensor is held by the base in accordance with a brain activity to be measured.

According to the physiological information measurement system described in Clause 6, the first sensor can be arranged at a position in accordance with a brain activity of interest so as to measure the brain activity.

### Clause 7

In the physiological information measurement system described in Clause 7, the signal processing unit is provided in the base.

According to the physiological information measurement system described in Clause 7, since the signal processing unit provided in the base can synchronize the first signal resulting from measurement by the first sensor with the second signal resulting from measurement by the second sensor, no other apparatus is required.

### Clause 8

In the physiological information measurement system described in Clause 8, the signal processing unit is provided in an apparatus different from the base.

According to the physiological information measurement system described in Clause 8, since an apparatus different from the base can synchronize the first signal resulting from measurement by the first sensor with the second signal resulting from measurement by the second sensor, the base can be lighter in weight.

### Clause 9

In the physiological information measurement system described in Clause 9, the signal processing unit synchronizes a first signal from the first sensor and a second signal from the second sensor with each other based on a time stamp of the first signal and a time stamp of the second signal.

According to the physiological information measurement system described in Clause 9, the first signal and the second signal can easily be synchronized with each other.

### Clause 10

In the physiological information measurement system described in Clause 10, the first sensor is a sensor that measures brain waves or a sensor that measures a cerebral blood flow, the second sensor is an image apparatus, and the physiological information measurement system further includes at least one sensor selected from the group consisting of a sensor that detects a motion of the subject, a sensor that measures a heartbeat, a sensor that measures a body temperature, and a sensor that measures respiration.

According to the physiological information measurement system described in Clause 10, various types of sensors can be adopted as the first sensor and the second sensor.

### Clause 11

In the physiological information measurement system described in Clause 11, the image apparatus is configured to obtain image of at least one of an operation performed by the subject and an environment around the subject as combined with a facial expression of the subject.

According to the physiological information measurement system described in Clause 11, a signal resulting from shooting of an operation performed by the subject or an environment around the subject other than the facial expression of the subject can be used.

### Clause 12

The physiological information measurement system described in Clause 12 further includes a voice collector that collects oral communication between the subject and a measurer.

According to the physiological information measurement system described in Clause 12, oral communication between the subject and the measurer collected by the voice collector can be used for measurement of brain activities.

### Clause 13

The physiological information measurement system described in Clause 13 further includes an audio device that presents an audio stimulus to the subject.

According to the physiological information measurement system described in Clause 13, brain activities at the time of presentation of the audio stimulus to the subject can be measured.

### Clause 14

The physiological information measurement system described in Clause 14 further includes a display device that presents a visual stimulus to the subject.

According to the physiological information measurement system described in Clause 14, brain activities at the time of presentation of the visual stimulus to the subject can be measured.

### Clause 15

The physiological information measurement system described in Clause 15 further includes a communication unit that communicates data with external equipment.

According to the physiological information measurement system described in Clause 15, the first signal and the second signal in synchronization with external equipment can be transmitted.

### Clause 16

A physiological information measurement system according to one aspect includes a base attachable to and removable from a head of a subject, a plurality of sensors held by the base, and a signal processing unit that processes signals outputted from the plurality of sensors. The plurality of sensors include a brain sensor that measures brain activities.

According to the physiological information measurement system described in Clause 16, a time period for attachment of a plurality of sensors to the subject and preparation for measurement can be reduced.

It should be understood that the embodiments disclosed herein are illustrative and non-restrictive in every respect. The scope of the present invention is defined by the terms of the claims rather than the description of the embodiments above and is intended to include any modifications within the scope and meaning equivalent to the terms of the claims.

### REFERENCE SIGNS LIST

1 head-mounted device; 2 signal processing apparatus; 10 housing; 11 vertex band; 12 head band; 13 size adjustment portion; 14 alignment portion; 15 receptacle; 16 control unit; 17 battery; 18 communication unit; 21 probe; 22 brain wave sensor; 31 ultra-wide-angle camera; 31a, 31b range of shooting; 32 temperature sensor; 33 acceleration sensor; 100, 100a head-mounted measurement system

1. A physiological information measurement system comprising:
a base attachable to and removable from a head of a subject;
a sensor held by the base, the sensor measuring brain activities;
a wide-angle camera that obtains an image of at least one of an operation performed by the subject and an environment around the subject as combined with a facial expression of the subject; and
a signal processing unit that synchronizes signals outputted from the sensor and the wide-angle camera.

2. The physiological information measurement system according to claim 1, wherein
the wide-angle camera is arranged at a position out of a field of view of the subject.

3. The physiological information measurement system according to claim 1, wherein
the base includes a protruding portion that protrudes forward from a frontal portion of the subject, and
the wide-angle camera is attached to the protruding portion.

4. The physiological information measurement system according to claim 1, comprising a fixing portion that removably fixes the base to the head of the subject, wherein
the fixing portion includes a vertex band for prevention of slip-off, a head band that fixes a position of the base, and a size adjustment portion that allows adjustment of a length of at least the head band in accordance with a size of the head.

5. The physiological information measurement system according to claim 4, wherein
the vertex band includes an alignment portion aligned with a vertex point.

6. The physiological information measurement system according to claim 1, wherein
the base includes a mechanism that changes a position where the first sensor is held by the base in accordance with a brain activity to be measured.

7. The physiological information measurement system according to claim 1, wherein
the signal processing unit is provided in the base.

8. The physiological information measurement system according to claim 1, wherein
the signal processing unit is provided in an apparatus different from the base.

9. The physiological information measurement system according to claim 1, wherein
the signal processing unit synchronizes a first signal from the first sensor and a second signal from the wide-angle camera with each other based on a time stamp of the first signal and a time stamp of the second signal.

10. The physiological information measurement system according to claim 1, wherein
the sensor measures brain waves or measures a cerebral blood flow,
and
the physiological information measurement system further comprises an additional sensor that performs at least one selected from the group consisting of detection of a motion of the subject, measurement of a heartbeat, measurement of a body temperature, and measurement of respiration.

11. The physiological information measurement system according to claim 10, wherein
the image apparatus is configured to obtain an image of at least one of an operation performed by the subject and an environment around the subject, as combined with a facial expression of the subject.

12. The physiological information measurement system according to claim 1, further comprising a voice collector that collects oral communication between the subject and a measurer.

13. The physiological information measurement system according to claim 1, further comprising an audio device that presents an audio stimulus to the subject.

14. The physiological information measurement system according to claim 1, further comprising a display device that presents a visual stimulus to the subject.

15. The physiological information measurement system according to claim 1, further comprising a communication unit that communicates data with external equipment.

16. A physiological information measurement system that measures brain activities, the physiological information measurement system comprising:
a base attachable to and removable from a head of a subject;
a plurality of sensors held by the base; and
a signal processing unit that processes signals outputted from the plurality of sensors, wherein
the plurality of sensors include a brain sensor that measures brain activities.
